(19) United States
(12) Patent Application Publication
(10) Pub. No.: US 2011/0125021 A1
(43) Pub. Date: May 26, 2011

(54) ACOUSTIC IMAGING APPARATUS WITH HANDS-FREE CONTROL

(75) Inventor: Wojtek Sudol, Andover, MA (US)

(73) Assignee: KONINKLIJKE PHILIPS ELECTRONICS N.V., Eindhoven (NL)

(21) Appl. No.: 13/056,157

(22) PCT Filed: Aug. 10, 2009
(86) PCT No.: PCT/IB2009/053515

§ 371 Date: Jan. 27, 2011

Related U.S. Application Data
(60) Provisional application No. 61/088,750, filed on Aug. 14, 2008.

(51) Int. Cl. A61B 8/14

(52) U.S. Cl. 600/443

(57) ABSTRACT

An acoustic imaging apparatus (100, 200, 300, 400) includes an acoustic probe (110) adapted to receive an acoustic signal, an acoustic signal processor (120) adapted to receive and process the acoustic signal from the acoustic probe, a display (130) for displaying images in response to the processed acoustic signal, and a non-manual control device (160, 160a, 160b, 160c). The acoustic imaging apparatus (100, 200, 300, 400) is adapted to control at least one of the acoustic probe (110), the acoustic signal processor (120) and the display 9130) in response to at least one signal from the non-manual control device (160, 160a, 160b, 160c). The non-manual control device (160, 160a, 160b, 160c) is either operated by a human foot, or mounted on a human head and operated by movement of the human head.

This invention pertains to acoustic imaging apparatuses, and more particularly to an acoustic imaging apparatus with hands-free control.

Acoustic waves (including, specifically, ultrasound) are useful in many scientific or technical fields, such as in medical diagnosis and medical procedures, non-destructive control of mechanical parts and underwater imaging, etc. Acoustic waves allow diagnoses and visualizations which are complementary to optical observations, because acoustic waves can travel in media that are not transparent to electromagnetic waves.

In one application, acoustic waves are employed by a medical practitioner in the course of performing a medical procedure. In particular, an acoustic imaging apparatus is employed to provide images of an area of interest to the medical practitioner to facilitate successful performance of the medical procedure.

One example of such a setting is a nerve block procedure. In such a procedure, an anesthesiologist controls an acoustic transducer of an acoustic imaging apparatus in one hand, and controls a needle in the other hand. Normally, the anesthesiologist makes all the adjustments to the acoustic imaging apparatus to get the desired picture before starting the procedure and before a sterile field is introduced.

However, often adjustments to the acoustic imaging apparatus are needed after the start of the nerve block procedure and/or after the area has been sterilized. Unfortunately, at that point, the anesthesiologist is not personally able to make further adjustments, and any adjustment must be made by an assistant or other person in response to instructions of the anesthesiologist. This can be awkward, cumbersome, and time consuming and can yield less than optimal results.

Other medical procedures can suffer from similar problems in the employment of acoustic imaging during the procedure.

Accordingly, it would be desirable to provide an acoustic imaging apparatus capable of hands-free control by a user.

In one aspect of the invention, an ultrasound imaging apparatus comprises: an ultrasound probe adapted to receive an ultrasound signal; an acoustic signal processor adapted to receive and process the ultrasound signal from the ultrasound probe; a display for displaying images in response to the processed ultrasound signal; and a control device that is adapted either to be operated by a human foot, or to be mounted on a human head and operated by movement of the human head, wherein the ultrasound imaging apparatus is adapted to control an operation of the acoustic probe, the acoustic signal processor, and/or the display in response to at least one signal from the control device.

In another aspect of the invention, an acoustic imaging apparatus comprises: an acoustic signal processor adapted to receive and process an acoustic signal received from an acoustic probe; a display for displaying images in response to the processed acoustic signal; and a non-manual control device, wherein the acoustic imaging apparatus is adapted to control an operation of the acoustic probe, the acoustic signal processor, and/or the display in response to at least one signal from the non-manual control device.

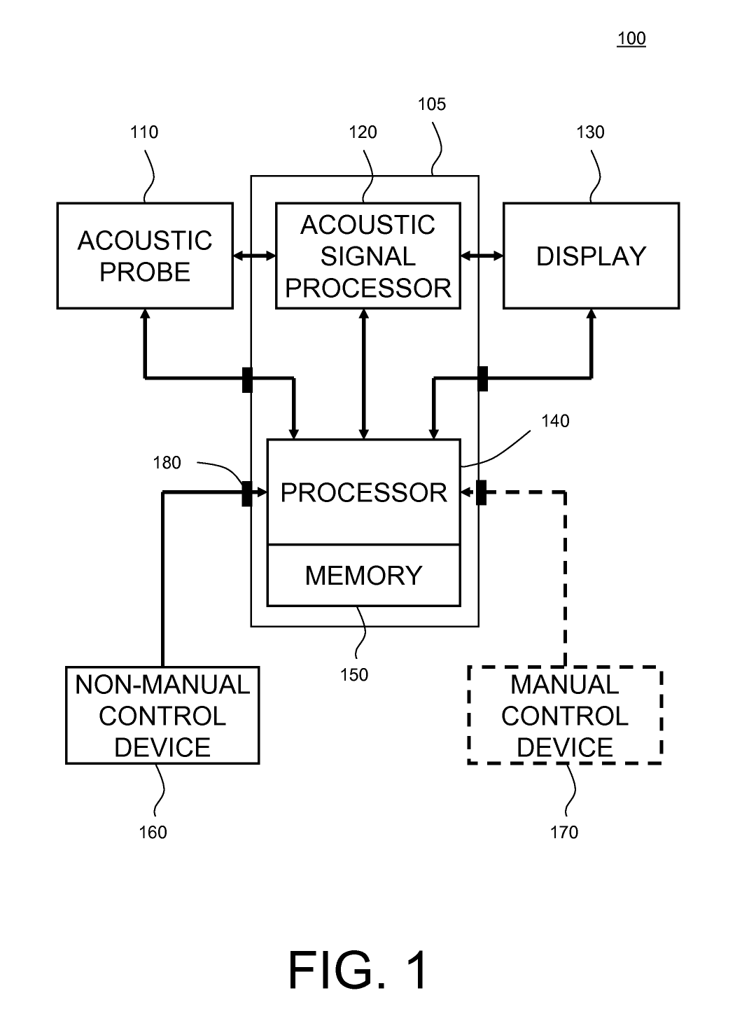

FIG. 1 is a block diagram of an acoustic imaging device.

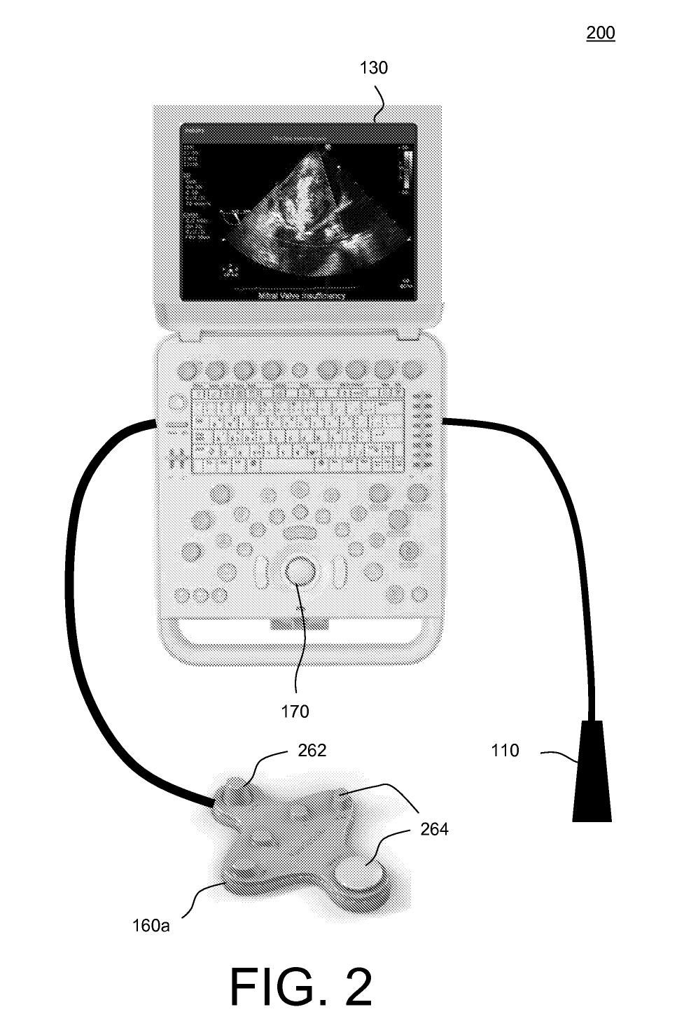

FIG. 2 illustrates one embodiment of the acoustic imaging device of FIG. 1.

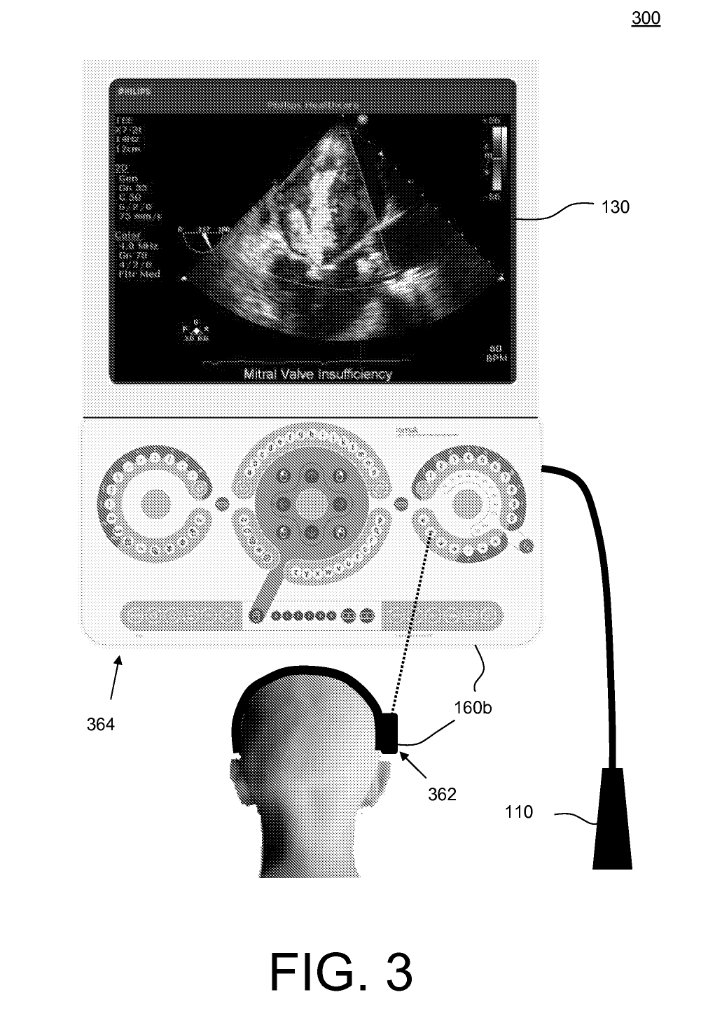

FIG. 3 illustrates another embodiment of the acoustic imaging device of FIG. 1.

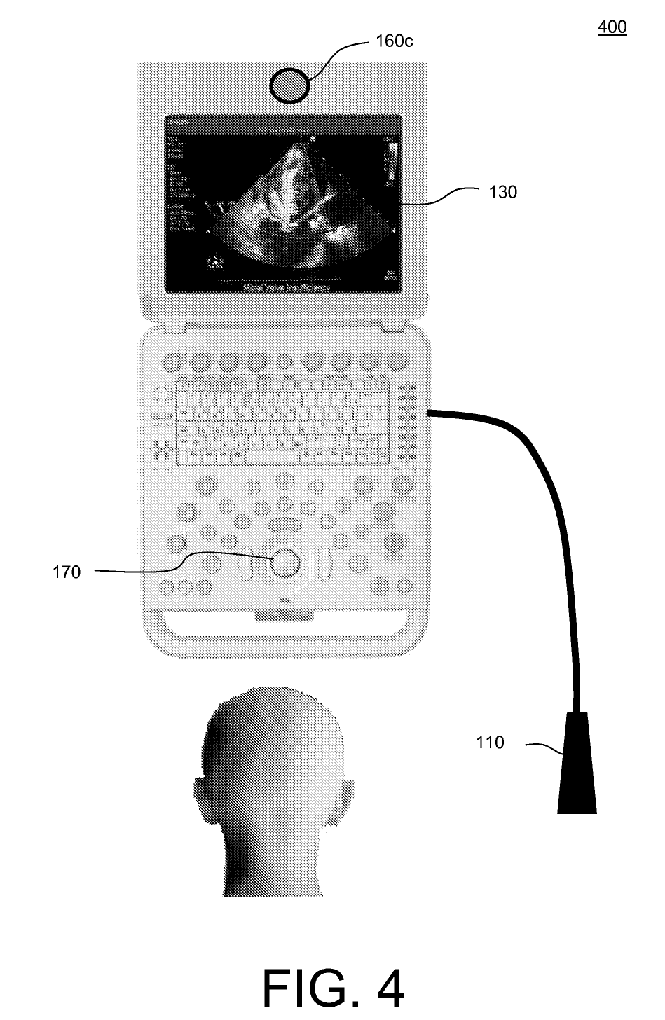

FIG. 4 illustrates yet another embodiment of the acoustic imaging device of FIG. 1.

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

As used herein, the term “non-manual control device” is defined as a device which can be controlled by a human user to produce a signal which may be used to control one or more operations of a processor-controlled apparatus, which device is adapted to respond to a movement of a part of the user's body, but which device is not adapted to be operated by a human hand. Non-limiting examples of such non-manual control devices will be described in greater detail below.

FIG. 1 is a high level functional block diagram of an acoustic imaging device 100. As will be appreciated by those skilled in the art, the various “parts” shown in FIG. 1 may be physically implemented using a software-controlled microprocessor, hard-wired logic circuits, or a combination thereof. Also, while the parts are functionally segregated in FIG. 1 for explanation purposes, they may be combined in various ways in any physical implementation.

Acoustic imaging device 100 includes an acoustic (e.g., ultrasound) probe 110, an acoustic (e.g., ultrasound) signal processor 120, a display 130, a processor 140, memory 150, a non-manual control device 160, and, optionally, a manual control device 170.

In acoustic imaging device 100, acoustic signal processor 120, processor 140, and memory 150 are provided in a common housing 105. However, display 130 may be provided in the same housing 105 as acoustic signal processor 120, processor 140, and memory 150. Furthermore, in some embodiments, housing 105 may include all of part of non-manual control device 160 and/or the optional manual control device 170 (where present). Other configurations are possible.

Acoustic probe 110 is adapted, at a minimum, to receive an acoustic signal. In one embodiment, acoustic probe is adapted to transmit an acoustic signal and to receive an acoustic “echo” produced by the transmitted acoustic signal.

In one embodiment, acoustic imaging device 100 may be provided without an integral acoustic probe 110, and instead may be adapted to operate with one or more varieties of acoustic probes which may be provided separately.

Processor 140 is configured to execute one or more software algorithms in conjunction with memory 150 to provide functionality for acoustic imaging apparatus 100. In one embodiment, processor executes a software algorithm to provide a graphical user interface to a user via display 130. Beneficially, processor 140 includes its own memory (e.g., nonvolatile memory) for storing executable software code that allows it to perform various functions of acoustic imaging apparatus 100. Alternatively, the executable code may be stored in designated memory locations within memory 150. Memory 150 also may store data in response to the processor 140.

Although acoustic imaging device 100 is illustrated in FIG. 1 as including processor 140 and a separate acoustic signal processor 120, in general, processor 140 and acoustic signal processor 120 may comprise any combination of hardware, firmware, and software. In particular, in one embodiment the operations of processor 140 and acoustic signal processor 120 may be performed by a single central processing unit (CPU). Many variations are possible consistent with the acoustic imaging device disclosed herein.

In one embodiment, processor 140 is configured to execute a software algorithm that provides, in conjunction with display 130, a graphical user interface to a user of acoustic imaging apparatus 100.

Input/output port(s) 180 facilitate communications between processor 140 and other devices. Input/output port(s) 180 may include one or more USB ports, Firewire ports, Bluetooth ports, wireless Ethernet ports, etc. In one embodiment, processor 140 receives one or more control signals from non-manual control device 160 via an input/output port 180.

As shown in FIG. 1, non-manual control device 160 is connected with processor 140 of acoustic imaging apparatus 100 via an input/output port 180. However, as explained above, in some embodiments, housing 105 may include all or part of non-manual control device 160. In that case, non-manual control device 160 is connected with processor 140 via internal connections or buses of acoustic imaging apparatus 100. Similarly, in FIG. 1 manual control device 170 is connected with processor 140 of acoustic imaging apparatus 100 via an input/output port 180. However, in some embodiments, manual control device 170 is connected with processor 140 via internal connections or buses of acoustic imaging apparatus 100.

Acoustic imaging apparatus 100 will now be explained in terms of an operation thereof. In particular, an exemplary operation of acoustic imaging apparatus 100 in conjunction with a nerve block procedure will now be explained.

Initially, a user (e.g., an anesthesiologist) makes all the adjustments to acoustic imaging apparatus 100 to get the desired picture before starting the procedure and before a sterile field is introduced. Such adjustments may be made via non-manual control device 160 or, beneficially, via manual control device 170 if present. When manual control device 170 is employed, then acoustic imaging apparatus 100 is adapted to control operation(s) of acoustic probe 110, acoustic signal processor 120, and/or display 130 in response to at least one signal from manual control device 170. Beneficially, when processor 140 is configured to execute a software algorithm that provides a graphical user interface to a user of acoustic imaging apparatus 100, then the user can navigate the graphical user interface via manual control device 170.

After acoustic imaging apparatus 100 is adjusted and the sterile field is introduced, then the anesthesiologist may manipulate the acoustic probe 110 with one hand and the needle with the other hand. At this time, acoustic probe 110 receives an acoustic (e.g., ultrasound) signal from a targeted region of a patient's body. Acoustic signal processor 120 receives and processes the acoustic signal from acoustic probe 110. Display 130 displays images of the targeted region of the patient's body in response to the processed acoustic signal.

Adjustments to acoustic imaging apparatus 100 may be needed after the start of the nerve block procedure and/or after the area has been sterilized. In that case, the anesthesiologist is capable of personally making further adjustments to acoustic imaging apparatus 100 via non-manual control device 160. Acoustic imaging apparatus 100 is adapted to control operation(s) of acoustic probe 110, acoustic signal processor 120, and/or display 130 in response to at least one signal from non-manual control device 160. Beneficially, when processor 140 is configured to execute a software algorithm that provides a graphical user interface to a user of acoustic imaging apparatus 100, then the anesthesiologist can navigate the graphical user interface via non-manual control device 160. Accordingly, adjustments to acoustic imaging apparatus 100 may be made by the anesthesiologist personally, without resorting to providing instructions or directions to an assistant.

Beneficially, non-manual control device 160 is adapted either to be operated by a human foot, or to be mounted on a human head and operated by movement of the human head.

FIG. 2 illustrates one embodiment of an acoustic imaging device 200. In acoustic imaging device 200, the non-manual control device is a foot-operated navigation device 160a. Foot-operated navigation device 160a includes a foot-operated joystick 262, and several buttons 264 that may be operated by a human foot. In operation, a user maneuvers foot-operated navigation device 160a with his/her foot. In response, foot-operated navigation device 160a provides a signal (e.g., to processor 140) which may be used for controlling an operation(s) of acoustic probe 110, acoustic signal processor 120, and/or display 130. When processor 140 is configured to execute a software algorithm that provides a graphical user interface to a user of acoustic imaging apparatus 200 via display 130, then the user can navigate the graphical user interface via foot-operated navigation device 160a.

FIG. 3 illustrates another embodiment of an acoustic imaging device 300. In acoustic imaging device 300, the non-manual control device is a head-mounted light operated navigation device 160b. Head-mounted light operated navigation device 160b includes a head-mounted light pointer 362 and a control pad 364. In one embodiment, head-mounted light pointer 362 includes a laser pointer, and control panel 364 includes a plurality of light-activated control pads. In operation, a user maneuvers his head to point a light beam (e.g., laser beam) from head-mounted light pointer 362 onto a desired control pad of control panel 364. In response, control panel 364 provides a signal (e.g., to processor 140) which may be used for controlling an operation(s) of acoustic probe 110, acoustic signal processor 120, and/or display 130. When processor 140 is configured to execute a software algorithm that provides a graphical user interface to a user of acoustic imaging apparatus 300 via display 130, then the user can navigate the graphical user interface via head-mounted light operated navigation device 160b.

FIG. 4 illustrates yet another embodiment of an acoustic imaging device 400. In acoustic imaging device 400, the non-manual control device is a head tracking pointer 160c. Head tracking pointer 160c includes a camera that produces a signal in response to a detected image of a human face. In particular, the camera operates with hardware and/or software to execute a facial recognition algorithm and to generate an output that depends upon an orientation of the human face whose image is captured by the camera. In operation, a user maneuvers his face to navigate a user interface via display 130 and the resulting camera output signal may be employed (e.g., together with a facial recognition algorithm) to control an operation(s) of acoustic probe 110, acoustic signal processor 120, and/or display 130.

Accordingly, as described above, an acoustic imaging device including a non-manual control device may be operated and controlled by a user in a hands-free manner. Furthermore, unlike systems that employ voice recognition, the acoustic imaging device having the non-manual control device can be controlled reliably by a user in applications and settings, such as operating rooms, where there may be many other people speaking and where there may be substantial background noise.

While preferred embodiments are disclosed herein, many variations are possible which remain within the concept and scope of the invention. Such variations would become clear to one of ordinary skill in the art after inspection of the specification, drawings and claims herein. The invention therefore is not to be restricted except within the spirit and scope of the appended claims.

1. An ultrasound imaging apparatus, comprising:
an ultrasound probe adapted to receive an ultrasound signal;
an acoustic signal processor adapted to receive and process the ultrasound signal from the ultrasound probe;
a manual control device for adjusting the ultrasound imaging apparatus prior to an imaging procedure;
a display for displaying images in response to the processed ultrasound signal; and
a control panel located adjacent to the ultrasound image display which includes a plurality of light activated controls;
a control device comprising a head-mounted light pointer that is adapted to be operated by movement of the human head to point a light beam onto a desired control of the control panel,
wherein the control panel provides a signal to control an operation of at least one of the ultrasound probe the acoustic signal processor and the display in response to to the pointing of a light beam onto a control of the control panel.

2. The ultrasound imaging apparatus of claim 1, wherein the control device is one of a head tracking pointer and a head-mounted light operated navigation device.

3. The ultrasound imaging apparatus of claim 1, further comprising a processor configured to execute a software algorithm, and that provides a graphical user interface to a user of the ultrasound imaging apparatus, and wherein the user can navigate the graphical user interface via the control device.

4. The ultrasound imaging apparatus of claim 3, wherein the manual control device is operable to navigate the graphical user interface via the manual control device, and wherein the manual control device includes at least one of a mouse, a joystick, and a trackball.

5. The ultrasound imaging apparatus of claim 3, wherein the processor is further adapted to control at least one of the acoustic probe, the acoustic signal processor and the display in response to at least one signal from the manual control device, and wherein the manual control device includes at least one of a mouse, a joystick, and a trackball.

6. (canceled)

7. The ultrasound imaging apparatus of claim 1, wherein the control device is a head tracking pointer.

8. The ultrasound imaging apparatus of claim 1, wherein the control device is a head-mounted light operated navigation device.

9-20. (canceled)
